(19) United States
(12) Patent Application Publication
(10) Pub. No.: US 2012/0232408 A1
(43) Pub. Date: Sep. 13, 2012

(54) Method and Apparatus for Cervical Cancer Screening

(76) Inventor: Laura Ann Weller-Brophy, Pittsford, NY (US)

(21) Appl. No.: 13/512,359

(22) PCT Filed: Nov. 17, 2010
(86) PCT No.: PCT/US10/56989

§ 371 Date: May 29, 2012

Related U.S. Application Data
(60) Provisional application No. 61/265,139, filed on Nov. 30, 2009.

(51) Int. Cl. A61B 6/00

(52) U.S. Cl. 600/478; 600/476

(57) ABSTRACT

The present invention relates to an apparatus for cervical cancer screening, comprising one or more light sources aligned with a beginning of a first optical test path and a beginning of a second optical test path, one or more optical detectors aligned with an end of the first optical test path and an end of the second optical test path, and a processor coupled to the one or more light sources and the one or more optical detectors and methods for using the same. The present invention further relates to a method for cervical cancer screening.

### FIELD OF THE INVENTION

The claimed invention relates generally to cervical cancer screening systems and methods, specifically to a light-based system and method for cervical cancer screening, and more specifically to an autofluorescence-based system and method for cervical cancer screening. Even more specifically, the invention pertains to an autofluorescence-based system and method for cervical cancer screening that illuminates tissues in certain embodiments at approximately the 290-310 nm band and includes screening of the cervical canal.

### BACKGROUND OF THE INVENTION

In the 60 years since its development, the Pap test has become the most common means for the screening of cervical neoplasia. The method's success is due to the qualities of low cost, minimal invasiveness, and ease of collection of the cervical cell sample. While use of Pap smears has produced a 74% drop in cervical cancer deaths in the US, there are limitations.

Pap smear sensitivity is low, ˜51%, and false negative rates for Pap tests range from 34-63% mainly due to inadequacies of sampling, slide preparation, or processing problems.

Following an abnormal Pap, a woman may be advised to have an additional Pap and/or a colposcopic examination with biopsy depending upon the severity of the Pap report. The colposcopic examination is a visual inspection of the cervix using an acetic acid application to highlight suspected abnormal tissue. The suspected abnormal sites are biopsied and sent for pathological review. The accuracy of this second level of screening and diagnosis is mixed. Approximately 200,000 women per year with highly significant precancerous conditions fail to be diagnosed by Pap smears followed by colposcopy; approximately 25% of patients seen for colposcopy do not follow-up as recommended, and colposcopy sensitivity is only about 67%. Fully ⅓ of high-grade disease is missed by initial colposcopy.

Most of the repeat Pap smears, colposcopic inspections, and biopsies confirm that the cervical tissues are normal. It is estimated that more than $10 million per day is spent unnecessarily on these procedures that could be eliminated if the Pap smear had a higher degree of sensitivity. These added procedures are estimated to add up to $3-6 billion yearly to US health care costs.

An added limitation of current methods of cervical cancer screening using Pap smears is the limited availability of pathology resources in under-developed regions of the world. While cervical cytologic testing has significantly reduced the cervical cancer death rate in developed countries, it remains unavailable in resource-poor regions of the world. Cervical cancer remains a leading cause of death for women outside of the US, with a death rate of about 500 women per day. In resource-poor regions of the world, the death rate results from the lack of cytologic screening facilities, insufficient numbers of trained personnel, and inability of patients to travel repeatedly to health facilities for the screening follow-up.

Current screening methods for cervical cancer commonly look for abnormal cells in a Pap smear in order to determine if a woman has cervical cancer or is at risk for cervical cancer. A woman is judged to be at risk for cervical cancer if she has cervical intraepithelial neoplasia (CIN). Cervical intraepithelial neoplasia is the potentially premalignant transformation and abnormal growth of squamous cells on the surface of the cervix. Also known as cervical dysplasia, CIN is not cervical cancer; most cases are resolved by a woman's immune system without medical treatment. Infection of the cervix by the sexually transmitted human papillomavirus (HPV) is the major cause of CIN. While CIN and cervical cancer can initiate in any portion of the cervix, they are most likely to occur in the transformation zone of the cervix. The transformation zone (referred to herein as TZ) is the area adjacent to the border of the endocervix and ectocervix and undergoes metaplasia numerous times during normal life. The TZ is a critical region for screening, as CIN lesions tend to occur in this area of the cervix. This is an area of dynamic change where the columnar epithelium transforms into squamous epithelium (the squamo-columnar junction). In younger women, the TZ tends to be located towards the exterior surface of the cervix. The TZ regresses into the cervical canal as a woman ages, and due to other circumstances such as child birth. When the whole squamo-columnar junction can be seen, the area at risk for CIN can be assessed with a colposcope, or other imaging method. If the squamo-columnar junction is in the cervical canal either partially or entirely, then the area at risk may not entirely. In this type of circumstance, an alternative or additional screening method is needed to fully diagnose cervical cancer. Traditionally, a physical cone biopsy with removal of a portion of the cervical canal may be indicated both for diagnosis and therapy.

The current cervical imaging methods for screening of cervical cancer, such as colposcopy and fluorescence imaging, direct light towards the cervical canal; neither method directly images or illuminates the cervical canal. Colposcopy and fluorescence imaging cannot be modified readily to allow simultaneous imaging of the surface of the cervical canal and of the surface of the cervix. The cervical canal is a tubular canal, typically several millimeters in diameter; such regions are most often viewed using endoscopic means. A large, fixed optical viewing system, such as a colposcope, designed to image the surface of the cervix, and not configured to be inserted into the cervical canal, will not provide the required interrogation of the cervical canal. In cases where the transformation zone has regressed either partially or fully into the cervical canal, current colposcopy and fluorescence methods will not be successful in the complete screening of the cervix for cancers and precancers.

Current imaging methods used in the screening of cervical cancer include colposcopy and fluorescence imaging. No commercial methods are available today that include imaging and light-based interrogation of the cervical canal. Fluorescence probes have been proposed for use in the interrogation of the cervical canal (U.S. Pat. No. 6,697,666, incorporated herein by reference), but have not been proposed for use together with cervical imaging methods including those using white light or fluorescence. In addition, the probes proposed for use in the endocervical canal have not controlled the per-point and total exposure of the illumination light. With the endocervical canal being an area of the body that is difficult to view, it is also desirable to remotely control the areas of the canal being exposed to light in addition to the total light exposure.

U.S. Pat. No. 5,773,835, published Jun. 30, 1998 and incorporated herein, discloses methods and devices for practicing fluorescence spectroscopy that employ a transparent fluoropolymer in the probe to reduce background fluorescence. A fiber probe apparatus is also disclosed that can include a drive means for rotating the optical fiber to interrogate the tissue. Moreover, the optical fiber can also be translated within the instrument by the drive means for analysis at different axial locations. The inventors do not disclose a means to use rotation and translation with a fiber-based device, nor do they address how the probe motion may be automated so to control the exposure of the tissue under test. There remains a need for a device and method for practicing fluorescence spectroscopy that allows the cervical canal to be interrogated, with a controlled exposure of the cervical canal by the illumination light.

U.S. Pat. No. 5,773,835 further describes an apparatus or system for use with an endoscope. The system can include imaging optics for viewing or recording images of the target tissue and/or observed fluorescence and for recording a permanent image of the tissue structures. The viewing/imaging module, as depicted by the inventors, consists of an undisclosed imager that is conducted through and integral to the tubular body of the endoscope. The authors do not teach the use of separately optimized probes for assessing a tubular structure and separately imaging an outer surface of that structure. In the case of the analysis of cervical cancer, and the need to assess both the relatively narrow cervical canal as well as the outer surface of the cervix, the small tubular probe required to fit into the cervical canal would not be adequately large to enclose the imaging optics required to take a high quality image of the cervical surface. U.S. Pat. No. 5,773,835 neither teaches a two-probe system, nor does it teach a method wherein a system might be used to both screen the cervical canal and image the outer surface of the cervix.

U.S. Pat. No. 6,949,072, issued to Furnish et al., Sep. 27, 2005 and incorporated herein, discloses devices for detecting plaque in arteries. In specific, the devices consist of a combination of infrared spectroscopy and intravascular ultrasound, integrated into a single probe. The intravascular probe can include one or more optical waveguides. In some embodiments, the intravascular probe includes a rotatable cable surrounding an optical waveguide, with the rotatable cable configured to coaxially rotate a beam director; the direction of the illumination beam around the artery wall is enabled by the rotation of a beam director and not by the optical fiber itself. This requires a probe of sufficient size to incorporate a mirror or other reflective optic at the tip of the probe, with a platform on which the mirror or other reflective optic might be mounted. The inventors do not teach a means to rotate the illuminating beam that does not require optical components in addition to the one or more optical fibers.

U.S. Pat. No. 7,261,687, issued to Yang, Aug. 28, 2007 and incorporated herein, provides a forward-imaging endoscope based upon Optical Coherence Tomography. The optical apparatus includes a refractive lens element near the end of an optical fiber at the tip of the endoscope; the lens element and optical fiber are both configured to rotate. In another embodiment, two lens elements are used with the optical fiber, with the two refractive lens elements configured to rotate about an axis. Scanning of the illumination beam is enabled by the rotation of one or more lenses. This requires a probe of sufficient size to incorporate a beam deflector and one or more lenses, with a support on which the optics might be mounted. The inventors do not teach a means to rotate the illuminating beam that does not require optical components in addition to the one or more optical fibers.

US 2009/0203991, with an Aug. 13, 2009 publication date and incorporated herein, is directed to the probing of atherosclerotic cardiovascular disease. In specific, the inventors teach the integration of a spectroscopic function with an ultrasound imaging function into a single probe. The applicants do not teach a system that forms both an optical image of a surface as well as the collection of fluorescence from a tubular structure. The applicants do not teach a two-probe system, nor do they teach a method wherein a system might be used to screen the cervical canal and to image the outer surface of the cervix. There remains a need for a two-probe system that can screen the cervical canal and the surface of the cervix for cervical cancer. Additionally, there remains a need for a two-probe system that can fully screen for cervical cancer, wherein the illumination of the cervical canal is controlled.

The use of UV-excited fluorescence to screen for gynecological cancers was pioneered by Leiner, who measured fluorescence spectra of sera from patients with gynecological malignancies. (Leiner, Marc J. P., Rudolf J. Schaur, Gernot Desoye, and Otto S. Wolfbels, “Fluorescence Topography in Biology. ill: Characteristic Deviations of Tryptophan Fluorescence in Sera of Patients with Gynecological Tumors,” Coo Chern 1986; 32(10):1974-1978, incorporated herein by reference.) Illumination of the sera at 287 nm efficiently excited tryptophan emission, with a major emission peak around 337 nm, and a smaller emission around 365 nm. The fluorescence spectra of sera from 19 healthy donors were compared with those from 31 patients having gynecological malignancies. The sera were illuminated at 287 nm, with fluorescence signals measured for each sample at 337 nm and 365 nm. A diagnostic ratio was formed by dividing the 365 nm fluorescence relative to the 337 nm signal. The mean diagnostic ratio from the healthy control group was 71.4 (Standard Deviation (SD) 0.8), significantly lower (p<0.001) than the mean value of the tumor group, 68.6 (SD 1.8). It was shown that the measured fluorescence was not due to tissue inflammation. This was confirmed by evaluating the fluorescence values for sera from patients with/without salpingitis. The salpingitis inflammation did not lead to changes in the fluorescence excited at 287 nm.

The first study of UV-excited fluorescence spectroscopy from ex vivo gynecological tissue was presented by Glassman (Glassman, W. Sha, C H. Liu, G C Tang, S Lubics, and R R Alfano, “Ultraviolet Excited fluorescence Spectra from Non-malignant and Malignant Tissues of the Gynecological Tract,” Lasers in the Life Sciences 1992; 5(1-2):49-58, incorporated herein by reference). Malignant and non-malignant tissues were obtained with each separated into two sections. The sections were submitted either for pathology or fluorescence measurement. A total of 22 malignant and 10 nonmalignant samples were measured. When illuminated at 300 nm, the samples displayed large fluorescence peaks around 340 nm (tryptophan fluorescence) and secondary peaks around 460-480 nm (NADH, elastin, and collagen fluorescence). A plot of the fluorescence spectrum of a non-malignant ovarian tissue is shown in FIG. 1, with the spectra at 340 nm and 440 nm identified by the dashed lines. As can be seen in FIG. 1, the 340 nm peak due to tryptophan is clearly evident, with sub-peaks noted around 460 nm, 490 nm, and 510-530 nm. The identification of the peaks and sub-peaks from FIG. 1 may be assisted by reference to FIG. 2. In this figure are shown two sets of plots. The upper plot depicts the absorption spectra of common fluorophores native to human and animal tissues. As shown in this upper plot, tryptophan, flavins, NAD+, elastin, collagen, and NADH all absorb light around 290-310 nm. These fluorophores emit light as indicated in the lower plot. This plot depicts the fluorescence spectra emitted by each of the fluorophores. As shown, tryptophan emits strongly around 340 nm. The spectra shown in this plot suggest that the 490-530 nm sub-peaks may result from flavins.

A diagnostic ratio was formed by dividing the measured fluorescence at 340 nm by that at 440 nm. It was found that 21 out of the 22 malignant samples had diagnostic ratios >12. All of the non-malignant tissue had diagnostic ratios <11. The difference of the two sets of diagnostic ratios allowed a separation of 21 of the malignant samples from the 10 non-malignant samples.

The measurements are shown in the first two rows of Table 1. The 300 nm illumination of gynecological tissues was able to differentiate cancerous from noncancerous tissues with a sensitivity and specificity of 9.5-100%.

Huang reported an experimental study, where 65 cancerous and 24 normal gynecological tissues were measured. See Huang, Z. Z., W. Sha. Glassman, G. C. Tang, S. Lubics, and R R Alfano, “Fluorescence Piagnosis of Gynecological Cancerous and Normal Tissues,” Proceedings of Advances in Laser and Light Spectroscopy to Diagnose Cancer and Other Diseases. SPIE 1994; 2135:42-45, incorporated herein by reference. The samples were separated into 2 pieces, with one piece used for pathological examination and the second used for fluorescence measurement. The diagnostic ratio of the measured fluorescence values at 340 nm/440 nm was formed for each measurement of the tissue samples. A diagnostic ration of 11.5 was used to separate the tissue types into cancerous (ratio>11.5) and normal (ratio<11.5). Of the 65 cancerous tissues, 63 had diagnostic ratios >11.5. Of the 24 normal tissues, 21 had diagnostic ratios <11.5. The corresponding false negative and false positive rates were 3.1% and 12.5%, respectively. The sensitivity and specificity are summarized in the last 2 rows of Table 1. Similar methods are cited in the expired U.S. Pat. No. 5,131,398, issued to Alfano et. al, Jul. 21, 1992.

TABLE 1
Performance of the 300 nm Fluorescence Excitation
method in distinguishing cancerous from noncancerous
gynecological tissues.

| Tissue Type | Number of | | |
|---|---|---|---|
| (in vitro) | Samples | Sensitivity | Specificity |
| GYN | 22 cancer | 95% | 100% |
| GYN | 10 noncancer | 95% | 100% |
| GYN | 65 cancer | 97% | 97% |
| GYN | 24 noncancer | 87.5% | 87.5% |

Results of Glassman reported in the first 2 rows, with the results of Huang in the last 2 rows.

Building on these results, Katz demonstrated the use of 2-D fluorescence imaging of ex vivo gynecological tissues to obtain a visual image of normal and cancerous regions. See Katz, Alan, Howard E. Savage, et al, “Ultraviolet and blue 2-D fluorescence snapping of gynecological tissues,” Optical Biopsy III, Proceedings of SPIE 2000; 3917:200-203, incorporated herein by reference. Multiple combinations of illumination and fluorescence wavelengths were used to create an image that allowed researchers to analyze the size, shape and location of different tissue structures. They were able to distinguish cancer from benign tissue by differences in the strength of fluorescence emission.

In summary, existing technologies that detect the threshold of high-grade cervical neoplasias (CIN 2, CIN 3, Carcinoma in Situ (CIS), and Invasive Cancer) include the Pap smear, colposcopy, and the Pap smear used with HPV DNA testing. These currently used methods all require samples to be collected and sent to a separate pathology laboratory where they are prepared for assessment and then screened for disease and disease precursors. Accordingly, these currently methods do not provide immediate results, require multiple collection and preparation steps for each sample, and depend upon trained pathology specialists to provide review of the pathology status. Given the multiple collection and preparation steps, as well as the human interpretation of the samples, errors may occur, with sensitivity of the Pap smear method about 50%. This low sensitivity requires screening redundancy and added procedures including colposcopy and physical biopsy to diagnose cervical cancer; most of the repeat Pap smears, colposcopic inspections, and biopsies confirm that the cervical tissues are normal. The addition of colposcopy and/or HPV DNA testing increases the sensitivity substantially (to over 90%), but at a cost of a drastic lowering of specificity to less than 50%. A low specificity means that the method may suffer from a significant number of false positive results, indicating that a patient has cervical cancer or precursors of cervical cancer when, in fact, their tissues are normal. False positive results lead to over-treatment of patients, unnecessary medical procedures, and health risks. These added procedures are estimated to add up to S3-6 billion yearly to US health care costs. In resource-poor countries, where trained pathologists and local health care facilities are rare, women do not have access to Pap smears. In such areas of the world, cervical cancer remains a leading cause of death for women, with about 500 women dying of the disease each day.

In comparison to the current standards of care, fluorescence-based methods offer immediate results, with no need for removal of tissues or availability of trained pathologists. Current fluorescence methods, however, are limited by relatively low specificity due to illumination wavelengths that excite confounding tissue fluorescence from inflamed tissue sites. In addition, current methods and equipment fail to interrogate and screen both the outer surface of the cervix as well as the cervical canal. As described herein, fluorescence sensing equipment and methods are detailed that facilitate interrogation and screening of both the outer surface of the cervix and the cervical canal; this is done through a single fluorescence-based system that comprises two probes.

### Problem to be Solved

There remains a need for a cervical cancer screening method having both sensitivity and specificity >80%, at a reduced cost relative to existing screening/diagnostic methods, that can be deployed by health care workers with limited training, and which provides immediate results so that treatment may be offered at the time of the test. A cervical cancer screening system is needed that incorporates probes that can be used to interrogate both the surface of the cervix as well as the cervical canal. In addition, a cervical cancer screening system is needed that controls the exposure to illumination light. Preferably, a cervical cancer screening system is needed that is relatively compact, with economical use of the most bulky and costly components so to facilitate a cost-effective system for use in diverse regions of the globe.

### SUMMARY OF THE INVENTION

This invention, relates to an apparatus for cervical cancer screening, comprising one or more light sources aligned with a beginning of a first optical test path and a beginning of a second optical test path; one or more optical detectors aligned with an end of the first optical test path and an end of the second optical test path; and a processor coupled to the one or more light sources and the one or more optical detectors.

The invention further relates to an apparatus for cervical cancer screening using two optical probes, comprising an imaging probe used to form an image primarily of the outer portion of the cervix, and a second probe used to illuminate and to collect light from the cervical canal.

In addition, the invention relates to a method for cervical cancer screening using two optical probes, comprising an imaging probe used to form an image primarily of the outer portion of the cervix, and a second probe used to illuminate and to collect light from the cervical canal.

The invention also relates to a method for cervical cancer screening using two optical probes, comprising the following steps;

a) insertion of an imaging probe into the vagina with white light, reflection, and/or fluorescence imaging of the cervix;

b) removal of the imaging probe;

c) insertion of the cervical canal probe into the cervical canal;

d) screening of the cervical canal; and

e) removal of the cervical canal probe.

The invention further relates to a method for cervical cancer screening using two optical probes, comprising the following steps:

a) insertion of a speculum into the vagina;

b) insertion of an imaging probe into the vagina with white light, reflection, and/or fluorescence imaging of the cervix;

c) removal of the imaging probe;

d) attachment of the attachment cap to the imaging probe;

e) insertion of the cervical canal probe into the cervical canal;

f) screening of the cervical canal;

g) removal of the cervical canal probe; and

h) removal of the speculum from the vagina.

Additionally, the invention relates to a method of screening the cervical canal for cervical cancer wherein one or more mechanisms are used to control the positioning of the probe interior to the endocervical canal, such mechanisms including, but not limited to an interior screw element and an exterior screw body that mate.

The invention further relates to an apparatus to screen the cervical canal comprising a fiber rotation means and a longitudinal translation means.

### Advantageous Effect of the Invention

The present invention includes several advantages, not all of which are incorporated in a single embodiment. The present cervical cancer screening method and apparatus offers both sensitivity and specificity >80%, and provides immediate results so that treatment may be offered at the time of the test. The present cervical cancer screening method and apparatus fully screens both the surface of the cervix and the endocervical canal, wherein both the surface of the cervix and the endocervical canal are interrogated through illumination, for example, in the approximately 290-310 nm band. This band is selected in some embodiments, as it has been shown to produce fluorescence correlated with cervical cancer disease state and to reduce confounding fluorescence signals from inflamed, but otherwise normal cervical tissues. In addition, the components of the imaging and cervical canal probe systems are integrated or shared to reduce overall cost or size of the system. Unlike conventional Pap smears, HPV DNA tests, and colposcopic evaluations, the present cervical cancer screening method and apparatus offers an immediate result, enabling the patient to receive follow-on care at the same visit, if necessary. The present cervical cancer screening method and apparatus eliminate the need for separate pathological confirmation of disease, although such confirmation may be used in conjunction with the present method, if desired. The present invention is advantaged over other fluorescence-based methods in that it preferentially uses the 290-310 nm illumination band, interrogates and screens both the surface of the cervix and the cervical canal, incorporates a means to control the position of the probe in the cervical canal, can control the point-wise and total exposure of the cervical canal to illumination light, and includes an apparatus that provides for the sharing of components so to reduce overall apparatus size and cost.

### BRIEF DESCRIPTION OF THE DRAWINGS

FIG. 1 represents the fluorescence spectrum of human ovarian tissue when illuminated with light at 300 nm (Glassman, W. Sha, CH Liu, GC Tang, S Lubics, and RR Alfano, “Ultraviolet Excited Fluorescence Spectra from Non-malignant and Malignant Tissues of the Gynecological Tract,” Lasers in the Life Sciences 1992; 5(1-2):49-58, incorporated herein by reference).

FIG. 2 represents the absorption and fluorescence spectra of fluorophores that are commonly found in human/mammalian cells and tissues. Noteworthy are the fluorescence maxima of collagen, elastin, and NADH which occur around 380-390 nm, 410 nm, and 450 nm. (Katz, Alan, and Robert R Alfano, “Optical biopsy—Detecting cancer with light,” LEOS Newsletter February 1996, pp 6-8, incorporated herein by reference).

FIG. 3A represents a schematic of a dual probe apparatus for cervical cancer screening comprising two probes, with an imaging probe to screen the surface of the cervix and a second probe to screen the cervical canal. Major components, in addition to the probes, include the illuminator module, the detector(s) module, and the processor.

FIG. 3B represents a schematic of an illuminator module aligned with a beginning of a second optical test path 330.

FIG. 3C represents a schematic of an illuminator module aligned with a beginning of a first optical test path 320.

FIG. 4 represents a schematic of one embodiment of a point probe system.

FIG. 5 represents a schematic of a single fiber point probe having an angled fiber end face.

FIG. 6 represents a fiber probe having a means to control the position of the fibers relative to the body of the probe; the control mechanism utilizes an exterior screw body that mates with an interior screw element. A translation means may be included. Both the rotation and translation mechanisms can be automated to control the exposure of the illumination light so to limit the total exposure to the patient.

FIG. 7 represents a schematic of a cervical cancer imaging probe.

FIG. 8 represents a schematic of the imaging subassembly of a cervical cancer imaging probe.

FIG. 9A represents a schematic of an attachment cap used to couple a cervical canal probe to an imaging probe.

FIG. 9B represents a schematic of a coupled cervical canal probe and an imaging probe via an attachment cap.

FIG. 10 represents a representative calibration curve used in the analysis of measured spectral data.

FIG. 11 represents fluorescence data from a cervical tissue sample illuminated at 300 nm. The data are corrected for dark levels.

FIG. 12 represents the fluorescence data from FIG. 11 plotted with the relative fluorescence using a log scale. The data is taken from the dotted line data of FIG. 11.

### DETAILED DESCRIPTION OF THE INVENTION

In the following detailed description, for purposes of explanation and not limitation, exemplary embodiments disclosing specific details are set forth in order to provide a thorough understanding of the claimed invention. However, it will be apparent to one having ordinary skill in the art having had the benefit of the present disclosure, that the claimed invention may be practiced in other embodiments that depart from the specific details disclosed herein. Moreover, descriptions of well-known devices, methods and materials may be omitted so as to not obscure the description of the claimed invention.

The present invention relates to a method and apparatus for cervical cancer screening, using a non-invasive light-based technique that is conducive to safety and ease of use in both medical facilities as well as in remote care locations.

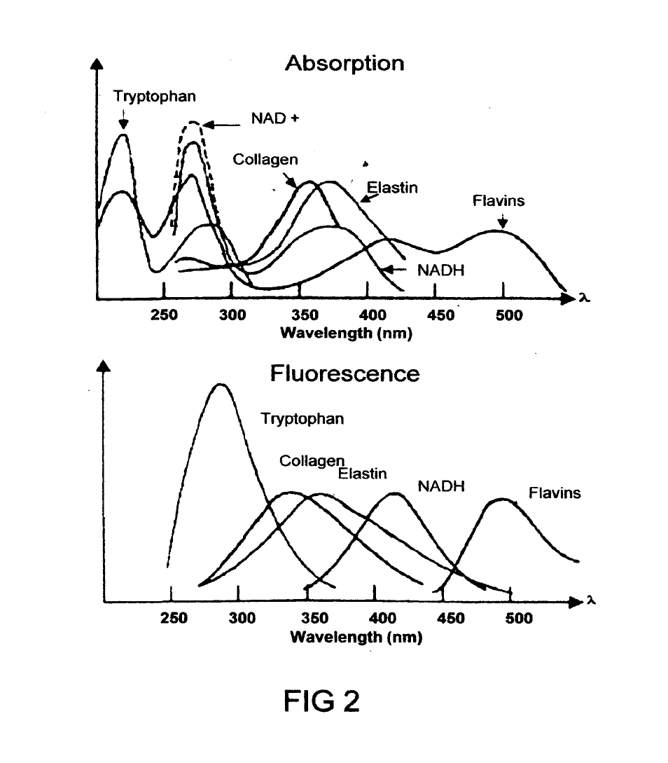

The use of tissue fluorescence to screen for biological abnormalities arises from the ability to excite autofluorescence from certain biological chromophores that are associated with disease progression. Identification of the excitation and emission spectra of such chromophores is based upon work conducted with isolated chromophores. Spectra of commonly measured chromophores are illustrated in FIG. 2. (Katz, Alan, and Robert R Alfano, “Optical biopsy-Detecting cancer with light,” LEOS Newsletter February 1996, pp 6-8, incorporated herein by reference.)

FIG. 2 illustrates the absorption and fluorescence spectra of fluorophores that are commonly found in human/mammalian cells and tissues. Noteworthy are the fluorescence maxima of collagen, elastin, and NADH which occur around 380-390 nm, 410 nm, and 450 nm. FIG. 2 indicates that illumination of a biological tissue with light approximately around 290-310 nm should most efficiently excite fluorescence from tryptophan (emission around 340 nm), and NAD+/NADH (emission around 460 nm).

From FIG. 2, it is apparent that illumination of biological tissues around 300 nm, 340 nm, or 400 nm will produce significant differences in fluorescence, as different biological chromophores are interrogated. While excitation around 300 nm preferentially excites tryptophan, illumination around 400 nm produces little excitation of this protein, with collagen, elastin, NADH, and flavins more efficiently excited. Illumination around 340 nm should effectively excite fluorescence emission from collagen and elastin (emission around 400 nm) and NADH (emission around 450 nm); tryptophan fluorescence is not excited. Experimental data indicate that the tryptophan fluorescence is more specific to cervical cancer identification than the fluorescence of collagen and elastin. While collagen and elastin fluorescence levels are correlated with cervical cancer, they also are indicative of inflammation, leading to the over-reporting of tissue inflammation as cervical cancer. While illumination/excitation wavelengths spanning 290-400 nm have been reported in the literature, the 290-310 nm illumination/excitation band is preferred in the methods and apparatus described herein, due the greater specificity of the excited fluorescence to screen for cervical cancer.

Cervical cancer screening requires both the screening of the surface of the cervix as well as the transformation zone. As a patient ages, the transformation zone regresses into the cervical canal, making it necessary to screen portions of the cervical canal in addition to the surface of the cervix.

Methods for cervical cancer screening disclosed here employ a novel two probe approach wherein an imaging probe is used to form an image primarily of the outer portion of the cervix. A second probe is used to collect light from the cervical canal. In a first embodiment, the image of the outer portion of the cervix is formed from reflected light. In a second embodiment, the image of the outer portion of the cervix is formed from fluorescence emitted from the cervical tissues. In a third embodiment, the images of the outer portion of the cervix are formed from both reflected light and fluorescence emitted from the cervical tissues.

In a further embodiment of the methods, the image of the outer surface of the cervix is formed in any of the first, second or third embodiments, with the second probe consisting of a non-imaging point probe that is used to scan the interior of the cervical canal. A point probe is an optical probe that illuminates a section of tissue, collecting light from that illuminated section; an image is not formed. In this case, the scanning of the interior of the cervical canal can be done using a reflected light signal, tissue fluorescence, or a combination of both reflected light and tissue fluorescence.

In yet another embodiment of the methods, the image of the outer surface of the cervix is formed in any of the first, second or third embodiments, with the second probe consisting of an imaging probe that is used to scan the interior of the cervical canal. In this case, the scanning of the interior of the cervical canal can be done using a reflected light signal, tissue fluorescence, or a combination of both reflected light and tissue fluorescence.

In a preferred embodiment of the methods, the image of the outer surface of the cervix is formed in any of the first, second, or third imaging embodiments, with the second probe consisting of a non-imaging point probe that is used to scan the interior of the cervical canal—the cervical canal probe. The scanning of the interior of the cervical canal is done by illumination of the tissues using light emitted by the probe in the 290-310 nm wavelength range. Fluorescence emitted by the cervical tissue is received by the probe and conducted to one or more detectors where specific fluorescence signals are measured in order to determine the disease state of the tissue under measurement. Successive patches of tissue are measured by moving the non-imaging point probe along sections of the cervical canal.

In a more preferred embodiment of the methods, the image of the outer surface of the cervix is formed by illumination of the cervix by a fluorescence imaging probe using light in the 290-310 nm range. Fluorescence emitted from the cervix is received by the fluorescence imaging probe and directed towards one or more image sensors. The one or more image sensors form images of the fluorescence emitted from the surface of the cervix. Following the fluorescence imaging of the surface of the cervix, the fluorescence imaging probe is removed from the vagina and a non-imaging fluorescence point probe is inserted therein. This point probe is guided into the endocervical canal to a depth of one or more cm, where it is used to collect fluorescence from successive patches of the endocervical canal. The scanning of the interior of the cervical canal is done by illumination of the tissues using light emitted by the probe in the 290-310 nm wavelength range. Fluorescence emitted by the cervical tissue is received by the non-imaging point probe and conducted to one or more detectors where specific fluorescence signals are measured in order to determine the disease state of the tissue under measurement. When the desired regions of the endocervical canal have been screened, the probe is removed from the body.

A method of screening may comprise the following steps; 1) insertion of speculum into the vagina to dilate it for examination of the cervix; 2) insertion of imaging probe through the speculum into the vagina with white light, reflection, and/or fluorescence imaging of the surface of the cervix; 3) removal of the imaging probe; 4) attachment of the attachment cap to the imaging probe; 5) insertion of the cervical canal probe into the cervical canal; 6) screening of the cervical canal; 7) removal of the cervical canal probe; 8) removal of the speculum from the vagina. Additional steps may be added and the order of steps may be altered as appropriate by those skilled in the art. Steps may be removed from the method; for example, the speculum may be eliminated from portions of the procedure where the imaging probe is used, as the imaging probe may be sufficiently large to dilate the vagina so that the cervix may be screened.

In addition to these methods are measurement systems and apparatus that include one or more measurement devices comprising the following elements:

One or more light sources that illuminates the tissue under examination. The light source may be one or more of an arc lamp with a narrowband filter, an LED with or without an optical filter, a fiber-coupled source, a laser, or another luminous emitter that has sufficient light energy in the desired optical band to excite fluorescence at the tissue site.

One or more optical detectors to measure the emitted fluorescence. The one or more detectors may be selected from diodes, CCDs, CMOS, with one or more photosensitive regions to sense the strength of emitted fluorescence. The one or more detectors can use one or more optical filters to select the specific wavelengths to be sensed. The optical filters may be cut-off filters, narrow-band filters, band edge filters, and can include beam splitters, plate filters, flexible filters, gratings, and other spectrally selective components known to those skilled in the art.

A probe body that serves to enclose the components that illuminate the tissue and that receive the fluorescence emitted by the tissue. Depending on the embodiment, the probe body may comprise plastic or metal materials, with transmissive windows and protective sheaths. The probe body may incorporate switching features to initiate a measurement.

A processor to run the measurement and to collect the measured data. The processor may be a computer, a laptop, a microprocessor, an application specific integrated circuit (ASIC), analog circuitry, digital circuitry, or any plurality and/or combination thereof configured to implement the disclosed methods and their equivalents. The processor may include or be configured to execute instructions stored on a computer readable storage medium for implementing the disclosed methods and their equivalents. Depending on the embodiments, configurations may include instructions to control variables such as exposure duration, illumination strength, total exposure, data collection and organization, data analysis, data review and display.

Additionally or optionally, the embodiments may include one or more couplers to connect, share, or couple components of the cervical canal probe with or to components of the imaging probe. Examples of couplers may include, but are not limited to, switches, an attachment cap, couplers, and other means.

Additionally or optionally, the embodiments may include a positioner to control the position of the cervical canal probe, including, as one example, a screw or other mechanical control so that the cervical canal probe does not overexpose regions of the cervical canal; the positioner may be mechanized so to automate the exposure when coupled to the illuminator.

Additionally or optionally, the embodiments may include a data communicator wherein data collected by the probe are transmitted to the processor and/or to another data receiver (for example, to a database). Such a data communicator may include, but is not limited to, a radio frequency (RF) transmitter and/or receiver. In other embodiments, data may be transmitted by satellite to enable remote data storage, analysis, retrieval, and other features.

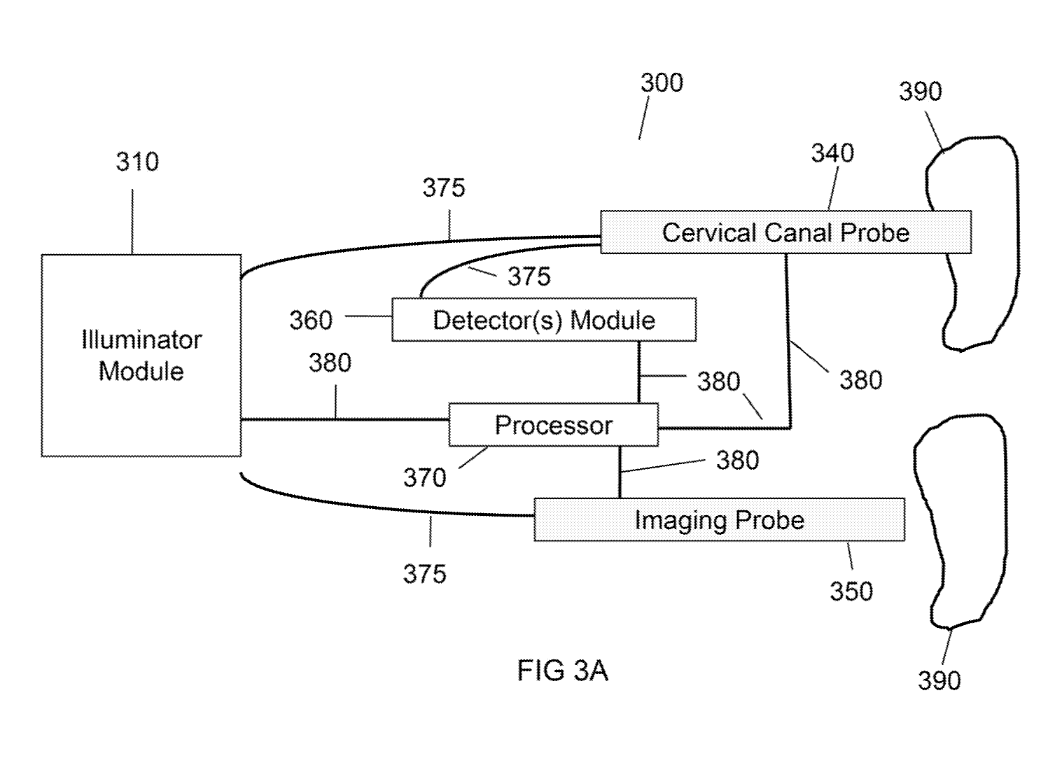

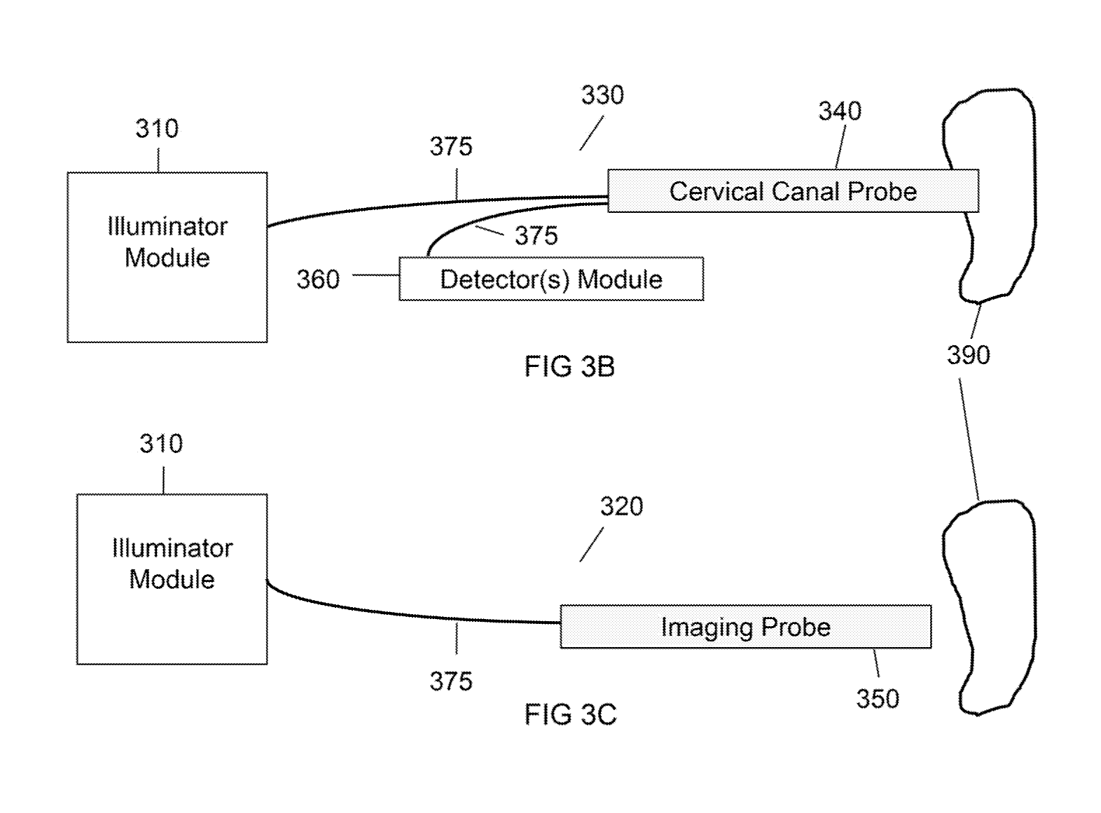

A dual probe apparatus for cervical cancer screening 300 is disclosed. The apparatus includes one or more light sources that are used to illuminate the cervical tissue under test. These one or more light sources are referred to as an Illuminator Module 310 as shown in FIG. 3. The light sources may be one or more of an arc lamp with a spectral filter, an LED with or without an optical filter, a fiber-coupled source, a laser, or another luminous emitter that has sufficient light energy in the desired optical band to excite fluorescence at the tissue site.

The one or more light sources are aligned with a beginning of a first optical test path 320 as shown in FIG. 3C and a beginning of a second optical test path 330 as shown in FIG. 3B. When optical sources are aligned, they are positioned so that the light emitted by the optical sources is directed towards an optical test path. An optical test path comprises the space and components that are placed between the one or more light sources and the cervical tissue under test 390. An optical test path may include optical lenses and/or mirrors to direct and to shape the light emitted by the one or more light sources, optical filters to control the spectral characteristics of the light emitted by the one or more light sources, windows and apertures to control the flow of light through the apparatus and to the tissue under test, optical fibers 375 to receive and to direct the light, and other components known to those skilled in the art. While optical fibers 375 are illustrated in FIG. 3, they may be replaced by, or used with other optical components known to those skilled in the art. Optical filters may include narrowband, notch, band-edge and other spectral filters used to shape the spectral content of the light emitted by the light source, as it interacts with the filter.

The apparatus for cervical cancer screening 300 further includes one or more optical detectors aligned with a portion of the first optical test path 320 and a portion of the second optical test path 330. As shown in FIG. 3A, the one or more optical detectors may be found in a detector module 360 where the detectors, filters, and associated optics may be co-located. One or more optical detectors may also be found within the probes 340 and/or 350. Optical detectors are aligned, or positioned, so that they receive light emitted by, or reflected by the tissue under test. The one or more detectors may be selected from diodes, Charge-Coupled Devices (CCDs), Complementary metal-oxide-semiconductor (CMOS) detectors, and light sensing elements with one or more photosensitive regions to sense the strength of emitted or reflected light. The one or more detectors can use one or more optical filters to select the specific wavelengths to be sensed. The optical filters may be cut-off filters, narrow-band filters, band edge filters, and can include beam splitters, plate filters, flexible filters, gratings, and other spectrally selective components known to those skilled in the art.

The apparatus 300 further comprises a processor 370 that may be coupled to the one or more light sources/illuminator module 310 and the one or more optical detectors including the detector module 360. The processor is coupled to the one or more light sources and/or the one or more optical detectors through wireless or wired connections 380 between the processor and the light sources and/or optical detectors. The wireless or wired connections allow electrical signals to be transferred between the processor and the light source, and/or the processor and the optical detectors. Similar wireless or wired connections 380 may also exist between the processor 370 and the probes 340 and 350 to control probe functions such as position, internal shutters, internal light sources, internal detectors, and other processor-controlled functions. The processor 370 may be a computer, a laptop, a microprocessor, an application specific integrated circuit (ASIC), analog circuitry, digital circuitry, or any plurality and/or combination thereof configured to implement the disclosed methods and their equivalents. The processor may include or be configured to execute instructions stored on a computer readable storage medium for implementing the disclosed methods and their equivalents. Depending on the embodiments, configurations may include instructions to control variables such as exposure duration, illumination strength, total exposure, data collection and organization, data analysis, data review and display. The processor may be a laptop computer with software developed to control the operation of the one or more light sources and detectors, allowing the illumination light to be controlled, reflected and/or fluorescence light to be detected, and detected signals to be analyzed. The function of the processor may be more complex, including timing of the one or more light sources, control of multiple output light signals, timing of the signals, coordination of the detectors to sense light reflected or emitted by the tissues under test, control of mechanical positioning included in the apparatus, analysis of the measured signals, and other functions known to those skilled in the art.

The apparatus may be configured so that the first optical test path 320 is configured to interrogate an outer portion of a cervix, and the second optical test path 330 is configured to interrogate an interior of a cervical canal. The first optical test path is configured to interrogate an outer portion of the cervix when the light emitted by one or more of the light sources is directed towards the outer portion of the cervix, illuminating the tissue under test. The first optical test path is further configured to interrogate an outer portion of the cervix when the light reflected or emitted by the tissue under test is directed towards the one or more optical detectors aligned with a portion of the first optical test path. Similarly, the second optical test path is configured to interrogate an interior portion of the cervical canal when the light emitted by one or more of the light sources is directed towards the inner portion of the cervical canal, illuminating the tissue under test. The second optical test path is further configured to interrogate an inner portion of the cervical canal when the light reflected or emitted by the tissue under test is directed towards the one or more optical detectors aligned with a portion of the second optical test path.

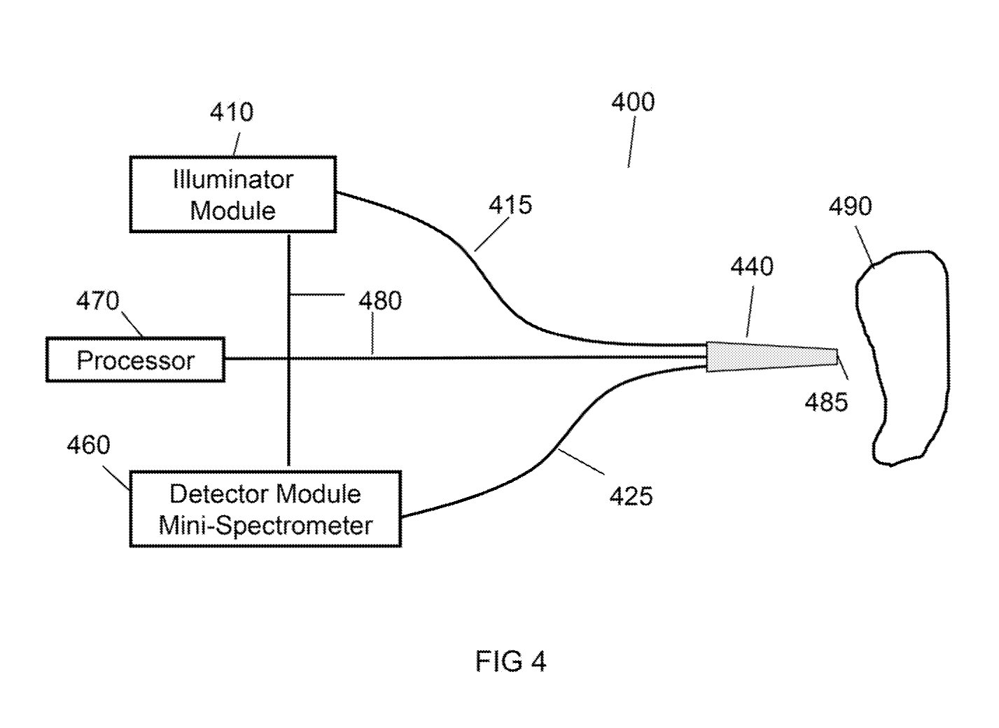

One embodiment of a point probe system 400 is illustrated in FIG. 4 and comprises an optical test path with a Mercury arc lamp illuminator module 410 directed towards and efficiently coupled to a 600 micron core silica fiber 415. The distal end of the fiber (the end of the fiber closest to the tissue under test 490) is used to illuminate the tissue under test 490. Fluorescence emitted from the tissue, as well as reflected illumination light, is captured by a second 600 micron silica fiber 425 that transmits this light to a detector module 360 (in this case, a Hamamatsu mini-spectrometer). The spectrometer is run by a processor 470, a laptop that controls data acquisition and saves the measured spectra. The processor 470 also may control the illuminator 410 via a shutter. The connections 480 of the processor 470 to the illuminator 410 and spectrometer 460 are electrical cables, but may be wireless connections if technically compatible. The processor 470 is shown with a connection 480 to the BioProbe 440. This connection is used to control data acquisition from the BioProbe 440 and can also be used to control illuminators and detectors housed within the BioProbe 440. The BioProbe 440 comprises a body that houses the lightguide 415 and collection fiber bundle 425. The body of the BioProbe 440 may be made of numerous biocompatible materials known to those skilled in the art, including surgical steel, and various plastics. The end of the BioProbe is covered by a window 485. Quartz is selected for its high transmission in the spectral ranges of interest; other materials may be used so long as optically transparent in the spectral ranges of interest, able to be cleaned according to accepted medical standards, and biocompatible with the tissues under test.

The end faces of the lightguide 415 and collection fibers 425 are located approximately 10 mm from the inner surface of the window 485. The quartz window is nominally 1 mm thick. With both the lightguide and collection fibers having the same core and clad dimensions, and the same numerical aperture (NA), they are preferably located at approximately the same distance from the tissue under test 490. For this apparatus, the end faces of the fibers were located about 5-12 mm from the surface under test. The end faces of the fiber may be placed closer to the quartz window (and to the surface under test). For example, they may be placed in near contact to the window, or several mm from the inner surface of the window. When the distance from the window is reduced, the illumination spot size is reduced. This leads to a higher density of illumination of the surface under test. When the distance of the illumination fiber to the window is increased, the energy density in the illumination spot decreases. Accordingly, the fiber-to-window distance may be used to change both the illumination spot size and the power density of the illuminating light. A range of illumination fiber-to-window and collection fiber-to-window distances may be used in other embodiments depending on the optics involved, the transmission of the probe, the wavelengths being used, and the sensitivity of the surface under test to the illuminating light.

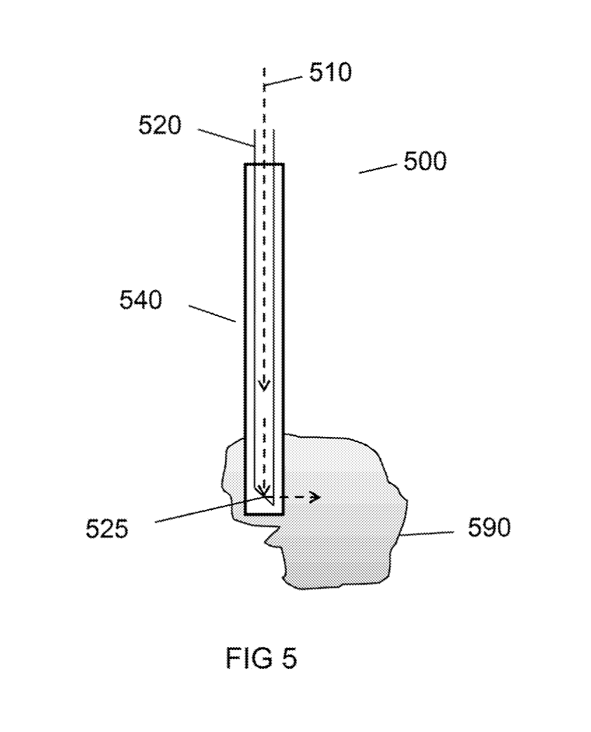

Point probes, of which the BioProbe of FIG. 4 is an example, can be made of diameters in the 3-4 mm range that comfortably fit the average cervical canal, with minimum diameters constrained by the components used to direct light to the tissue under test. A portion of a point probe is shown in FIG. 5, illustrating a single fiber example. In this figure, a portion of a point probe 500 is illustrated, comprising a single optical fiber 520 that is housed in a slender probe body 540. The end of the single optical fiber 520 that is closest to the tissue under test 590 may be finished with a flat end face that is perpendicular to the longitudinal axis of the fiber, or angled as shown in the figure by angled end facet 525. This end of the fiber may also be rounded or incorporate a lens that can shape and direct the illumination light as well as collect the reflected and or fluorescence emitted by the tissue under test 590. Light is coupled into the fiber by a remote illuminator, providing illumination light 510 to the tissue under test. For example, a single 600 micron fiber may be used to direct light to the tissue under test 590, with the same fiber serving to collect the fluorescence. A beam splitter at the far end of the fiber (not shown) may be used to separate the excitation and fluorescence paths.

A two fiber device can more efficiently handle the illumination and fluorescence optical paths, with reduced optical losses at the beam splitter, in this case one may employ two 600 micron (or other diameter) optical fibers, with each directed either to a light source or to a filter and detector for measurement of fluorescence. For a side-directed point probe for use in the cervical canal, the optical fibers may be cleaved at angles around 40 degrees so that the illumination light is totally internally reflected at the fiber cleave. In this manner, the light is emitted from the fiber nearly perpendicular to the fiber axis. Fluorescence emitted from the tissue under test is coupled into the fiber through the fiber cleave. Such a side-directed fiber probe arrangement allows tubular structures such as the cervical canal to be probed. This is the arrangement shown in FIG. 5 where the optical fiber 520 has an angle-cleaved end face 525 that is preferably polished. In the case where 2 fibers are used instead of one, both may be fit into a 2 mm outer diameter (OD) steel tube, protruding from the distal end (the end closest to the tissue under test). A quartz window can be fixed at this end to contain the fibers.

Other non-imaging probes are known in the art including ring probes wherein a ring of fibers are arrayed and used to collect or excite fluorescence. Also known are linear arrays of fibers, and other arrays or groups of fibers used to transmit illumination light and to collect fluorescence light, reflection light, and other light emitted by or reflected by the tissue under test.

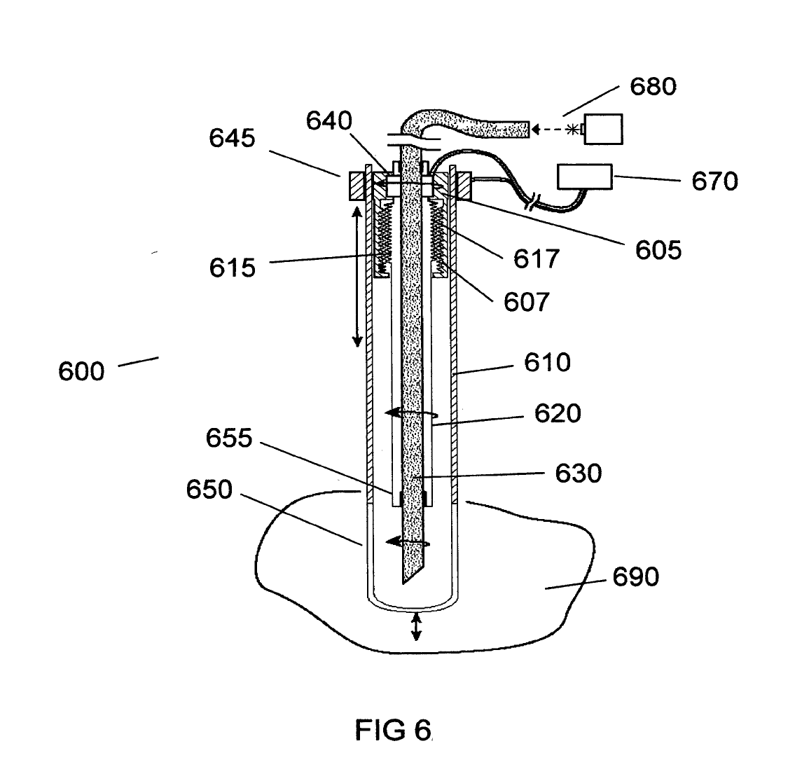

Control of the depth of insertion of the fiber probes may be done through the use of screw features or step features and translation means in mating tubes. The use of such features with automated processor control also may be used to limit or to fix the exposure of the tissue under test to the illumination light. In one embodiment, the mechanism used to control the depth of insertion comprises an interior screw element and an exterior screw body that mate. FIG. 6 illustrates a schematic of one fiber probe design. The fiber probe 600 comprises an exterior tube 610 and an interior tube 620, with the interior tube 620 housing a portion of an optical test path. In this example, the optical test path comprises, in part, an optical fiber 630. As illustrated in FIG. 6, an exterior screw body 605 includes an internal thread 607 cut into a portion of the exterior tube. Internal thread 607 mates with a male thread 617 that is cut on the interior screw element 615 of the interior tube 620. These mating threads allow the interior screw element 615 to be moved along a portion of the length of the exterior screw body 605. The interior tube 620 houses a portion of a first or second optical test path, directing light towards the tissue under test. The interior tube 620 may house one or more optical fibers, lenses, and filters to convey the illumination light towards the tissue under test, and to spectrally filter that light. Additionally, the interior tube 620 may house one or more optical fibers, lenses, and filters to receive fluorescence light emitted by the tissue under test, as well as incident light reflected by the tissue under test. The optical fibers, lenses, filters, and other optical components used to illuminate surfaces and to collect light, and known to those skilled in the art, are termed the optical illumination and collection optics of the point probe. One or more of the optical illumination and collection optics may be attached to the probe with methods known to those skilled in the art. The exterior tube 610 includes one or more transparent windows 650 that allow for transmission of both the illumination light and the fluorescence emitted from the tissue under test 690. The screw mechanism can be automated to control the exposure of the illumination light so to limit the total exposure to the patient. Other mechanical mechanisms are envisioned including step features, wedges and other elements that are known to those skilled in the art. For example, a 2 mm stainless steel interior tube 620 can be threaded at a region above the window 650 with this threaded region fit into a second larger bore exterior tube 610 with a mating thread. The entire assembly would be inserted into the cervical canal. Preferably, a stop (not shown) would be located at the entrance to the cervix so that the ends of the optical fiber 630 or fibers closest to the surface under test would be located at a known distance into the cervical canal. The thread or other mating features could then be rotated so to move the optical fiber or fibers 630 along the cervical canal through a predetermined pathway. If this movement is done in a motorized fashion, it can further act to control the optical exposure through interaction of the timed movement of the fibers with the optical exposure of the cervix. In cases where the exposure is in the UV, this serves to control the point wise and total UV exposure to the patient. The mating tube designs incorporate transparent windows so that the illumination and fluorescence beams are efficiently transmitted as the mechanical portions rotate or otherwise move into position. While rotation and thread screws are mentioned here as easily described means to scan a path in a tubular geometry, this implementation is provided as an example and not meant to limit those skilled in the art.

A preferred means to control the illumination light comprises an optical fiber rotation means that is coupled with a longitudinal translation means. As illustrated in FIG. 6, the fiber probe 600 comprises an interior tube 620 that houses a portion of a first or second optical test path, directing light towards the tissue under test. The interior tube 620 may house one or more optical fibers 630, lenses, and filters to convey the illumination light towards the tissue under test, and to spectrally filter that light. Additionally, the interior tube 620 may house one or more optical fibers, lenses, and filters to receive fluorescence light emitted by the tissue under test, as well as incident light reflected by the tissue under test. An exterior tube 610 is located around the interior tube 620. The exterior tube 610 includes one or more transparent windows 650 that allow for transmission of both the illumination light and the fluorescence emitted from the tissue under test. A rotation means 640 is coupled to the interior tube 620, with a longitudinal translation means 645 coupled to the exterior tube 610. The rotation means 640 causes the interior tube 620, with one or more attached optical fibers 630, to rotate at least one rotation around the longitudinal axis of the probe 600. As illustrated in FIG. 6, an optical fiber 630 located in the interior tube 620 forms a portion of an optical test path. The optical fiber 630 is adhesively attached to the interior tube 620 as illustrated by adhesive attachment points 655 near the end of the interior tube 620 closest to the tissue under test 690. Similar attachment points are located between the optical fiber 630 and the interior tube 620 near the top of the interior tube. Additional or other adhesive attachment points may be used. The adhesive attachment serves to hold the optical fiber 630 in place within the interior tube 620, provides strain relief to the fiber, and prevents fiber bends and twists within the interior tube. A longitudinal translation means 645, coupled to the exterior tube 610, controls the position of the optical fiber or fibers 630 within the body of the patient. Both the rotation and translation mechanisms can be automated to control the exposure of the illumination light so to limit the total exposure to the patient. Control may be achieved using one or more controllers 670 that are hard-wired to the rotation and translation mechanisms; the one or more controllers 670 may also be accessed through one or more wireless connections. Illumination light 680 is launched into the one or more optical fibers 630 using optical components known to those skilled in the art, including lenses, mirrors, optical filters, and similar components that shape, direct, and filter the illumination light.

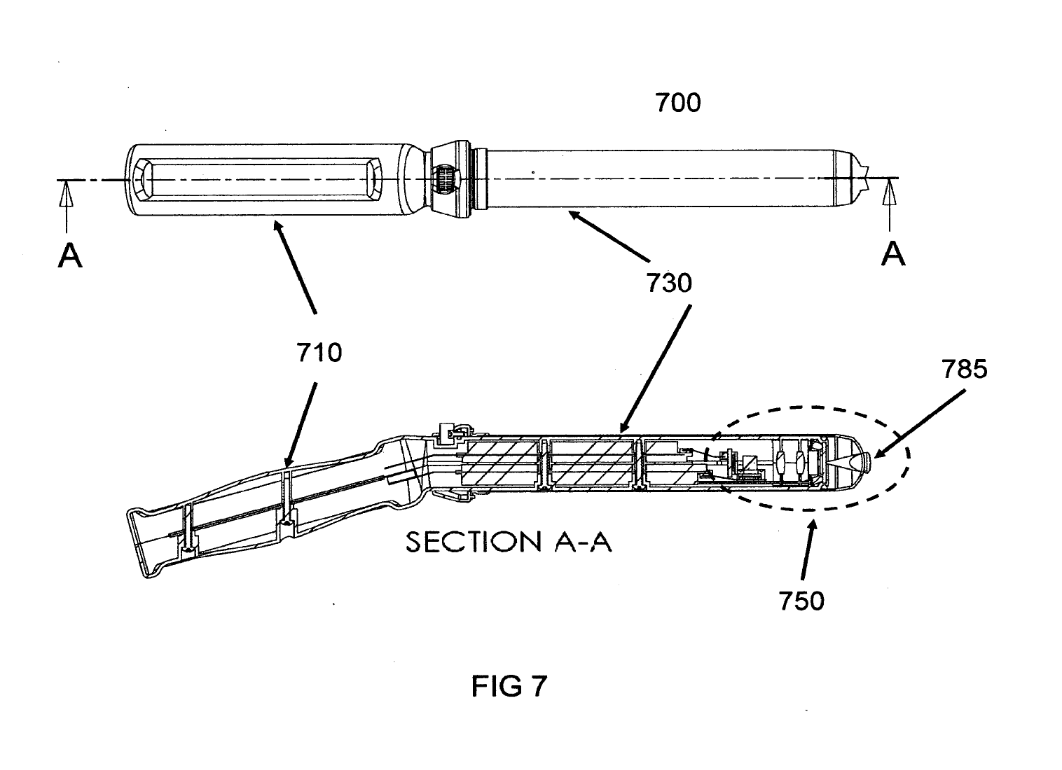

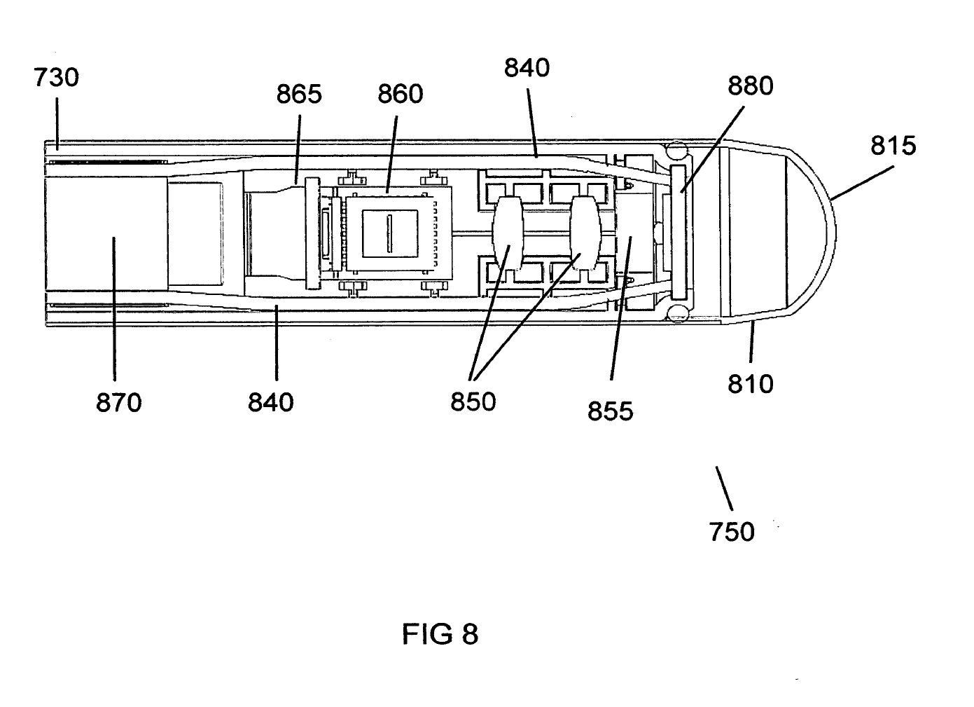

The full screening of the cervix entails both the screening of the cervical canal, as noted above, and the imaging of the exterior surface of the cervix. Imaging of the cervix may be done using a device as illustrated in FIG. 7. This figure shows a schematic of an imaging probe 700 in two views. The top view shows the probe from the top; the handle 710 is to the left, with the front of the probe to the right. The arrows at A-A show the cross-section cut. This cross-section is shown at the lower half of the figure. Again, the handle of the probe is to the left, with the front of the probe to the right. The front of the probe is inserted into the body of the patient, with the probe front 785 lightly touched to the patient's cervix. A protective sheath, shown in more detail in FIG. 8, is used to cover the imaging probe. Not shown is the illumination light source; this is located in an illumination module. Light from the illumination source is launched into a fiber cable that is put into the imaging probe 700. One or more fibers from the cable are routed to the front of the imaging probe where the light is transmitted by the probe front window and illuminates the tissue under test. The illuminated tissue fluoresces, with the fluorescence collected through the probe front 785. This light is imaged by the imaging subassembly 750, located in the imaging probe body 730. The probe body may be fabricated from a medically approved plastic or other materials so long as medically accepted, able to be cleaned according to accepted medical standards, and biocompatible with the tissues under test.

A schematic of one design of the imaging subassembly 750, including imaging and detection optics and electronics is shown in FIG. 8. This figure illustrates the imaging subassembly 750 with a protective sheath 810. The protective sheath is used to provide a physical barrier between the probe and the patient, with the sheath replaced by a sterile sheath for each use with a different patient. The sheath 810 includes a curved window 815 that assists in the imaging of the cervical tissues. The probe is located behind the sheath, with the front probe body 730 enclosed by the sheath 810. A flat quartz window 880 seals the front of the probe body 730. Behind the quartz window is an optical filter 855 followed by a two-lens imaging system 850. Behind the lens system 850 is an image sensor cube 860 comprising image sensors on different faces of the cube. Each of the sensors is covered by a different narrowband filter; a beam splitter is located within the cube to direct the optical image to the sensors. The image sensors are filtered by one or more narrowband optical filters that separate specific diagnostic wavelengths of light used in the screening of cervical cancer and precancers. The image sensor cube includes flex circuitry 865 that attaches the sensors to control electronics 870 in the probe body (to the left of the sensor cube). The control electronic boards 870 are interconnected to an external control/power module by electrical cables (not shown). Also shown in the figure are channels 840 that are used to house illumination fibers. These fibers conduct the 300 nm illumination light from an external light source to the front of the probe. The fibers terminate in ferrules (fiber and ferrules not shown) located behind the front filter 855.

The illuminated tissue fluoresces, with the fluorescence collected through the window 815 of the sheath 810, and the probe front window 880. This light is imaged by the optics 850, directed onto the image sensors located in the sensor cube 860.

Imaging probes as illustrated above may be limited in size due to the image sensors and associated electronics. For example, CCD sensors and associated electronics may limit the overall minimum diameter of the device to about 0.7-1.0 inch, and allow imaging of most of the surface of the cervix. Use of a CMOS sensor, with integrated electronics, may allow the overall minimum diameter to decrease to less than 0.5 inch. The length of the device is not as critical and may be varied depending upon the size of patient. Typical lengths may range from 10 inches to 18 inches, although both larger and smaller sizes may be needed in diverse populations.

Other imaging probes are known in the art including those that scan the cervix as well as those that are based on colposcopes. No reported imaging systems include both imaging and cervical canal probes.

A dual probe apparatus or system for cervical cancer screening is disclosed, comprising two probes that are used to excite and measure fluorescence from the cervix. The probes excite and measure fluorescence from both the outer surface of the surface as well as the cervical canal. The probes share common elements of equipment so to cause the overall system to be more compact in size and to reduce the overall cost of the system. The dual probe system can comprise a shared UV light source that emits light in a band including the 290-310 nm band. This light source can be switched between the imaging probe and the probe used to interrogate and screen the endocervical canal. The system can comprise a shared UV light source and also a coupling means to direct the output from the endocervical canal probe towards the imaging probe to use the same detectors, filters, and electronics as the imaging probe. In addition, the system can include a probe used to screen the endocervical canal that uses a mechanism to control the positioning of the probe interior to the endocervical canal. The apparatus comprises two probes that are used to excite and measure fluorescence from the cervix. The probes share common elements of equipment so to cause the overall system to be more compact in size and to reduce the overall cost of the system.

A first embodiment of the dual probe system comprises a shared UV light source that emits light in a band including the 290-310 nm band. This light source can be switched between the imaging probe and the probe used to screen the endocervical canal.

A second embodiment of the system comprises a shared UV light source and also a coupling means to direct the output from the endocervical canal probe towards the imaging probe to use the same detectors, filters, and electronics as the imaging probe.

A third embodiment of the system includes a probe used to screen the endocervical canal that uses a mechanism to control the positioning of the probe interior to the endocervical canal. In one embodiment, the mechanism comprises an interior screw element and an exterior screw body that mate. A method of screening the cervical canal for cervical cancer utilizes this third embodiment, wherein one or more mechanisms are used to control the positioning of the probe interior to the endocervical canal, such mechanisms including, but not limited to an interior screw element and an exterior screw body that mate. The exterior screw body includes one or more transparent windows that allow for transmission of both the illumination light and the fluorescence emitted from the tissue under test. The screw mechanism can be automated to control the exposure of the illumination light so to limit the total exposure to the patient. Other mechanical mechanisms are envisioned including longitudinal translation, step features, wedges and other elements that are known to those skilled in the art.

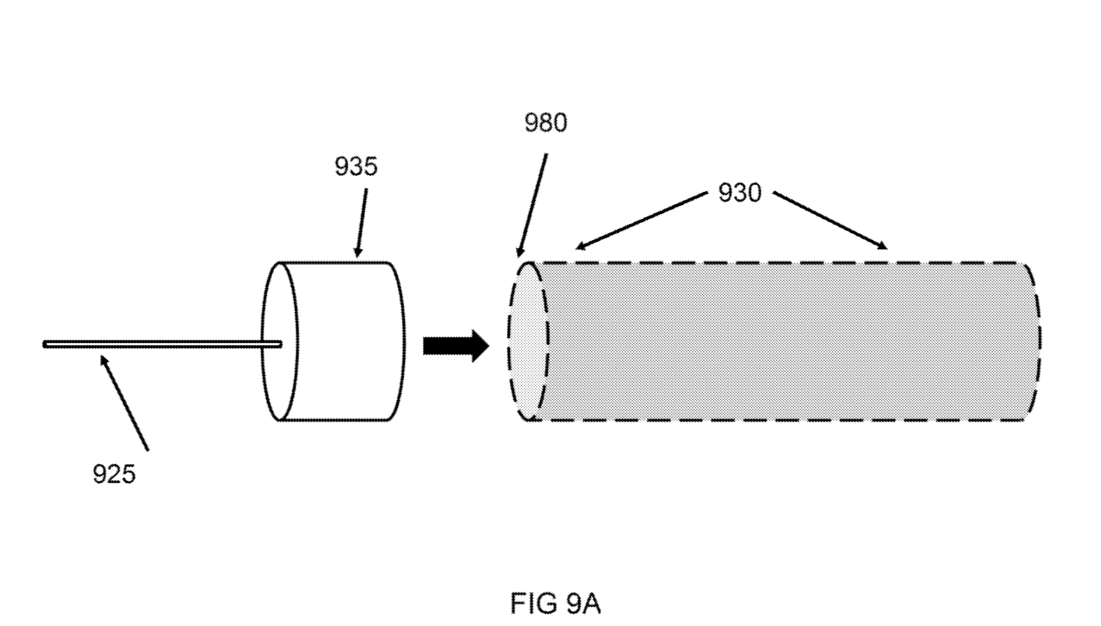

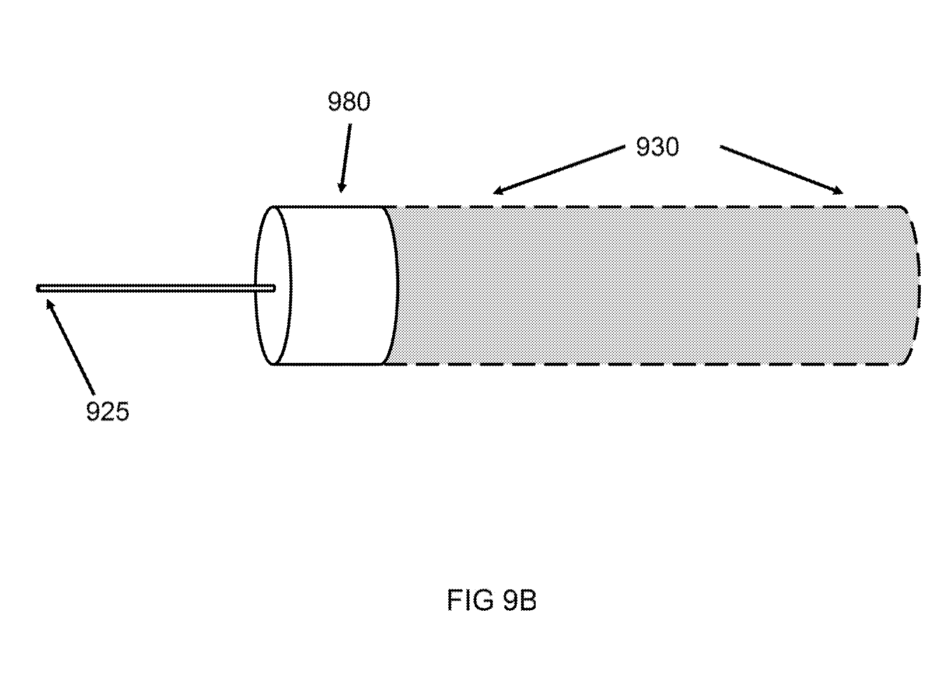

In a further embodiment of the system illustrated in FIGS. 9A and 9B, the probe used to screen the endocervical canal has an output fiber 925 that is terminated in an attachment cap 935. The output fiber carries the fluorescence signals that are emitted from the tissue under test. The attachment cap is fabricated to fit snugly over the front of the body 930 of the imaging probe. As shown in FIG. 9A, the attachment cap is brought towards the imagine probe where the window 980 is located, so that, as shown in FIG. 9B, it is fit snugly over the front of the imaging probe. The light emitted from the output fiber 925 passes through the quartz front window 980 and is captured by the optical elements within the imaging probe (see FIG. 8). The optical elements serve, for example, to direct the fluorescence to one or more image sensors and narrowband filters. In this manner, the optical system and associated electronics employed by the imaging probe are reused by the cervical canal probe by way of the attachment cap. While the FIG. 9 show the attachment cap 935 fitting over the body 930 of the probe, the attachment cap may also be fit over a sheath (not shown) that is fit over the body 930 of the probe.

The elements of these embodiments of the dual probe apparatus can be shared so to optimize the system performance, size, and cost.

Having thus described several embodiments of a method and apparatus for cervical cancer screening, it will be rather apparent to those skilled in the art that the foregoing detailed disclosure is intended to be presented by way of example only, and is not limiting. Various alterations, improvements, and modifications will occur and are intended to those skilled in the art, though not expressly stated herein. These alterations, improvements, and modifications are intended to be suggested hereby, and are within the spirit and the scope of the claimed invention. Additionally, the recited order of the processing elements or sequences, or the use of numbers, letters, or other designations therefore, is not intended to limit the claimed processes to any order except as may be specified in the claims. Accordingly, the claimed invention is limited only by the following claims and equivalents thereto.

### EXAMPLES

Table 1 compares existing technologies that detect the threshold of high-grade cervical neoplasias (CIN 2, CIN 3, CIS, and Invasive Cancer). Included in the table are experimental methods based upon cervical tissue fluorescence. The Guided Therapeutics data are from FDA trials. The 300 nm data are from published reports, and represent the results anticipated for a BioProbe according to the current invention.

TABLE 1
Summary of methods for the screening and diagnosis of
cervical cancer, together with the Guided Therapeutics
LightTouch ™ currently in FDA trials, and the
300 nm Fluorescence Excitation method proposed herein.

| Screening | | | Immediate | |
|---|---|---|---|---|
| Method | Sensitivity | Specificity | Result? | Turn-Around |
| Pap Smear¹ | 51% | 97% | No | 1-2 weeks |
| Colposcopy³ | 96% | 48% | No | 2-4 weeks |
| | | | | for biopsies |
| Pap + HPV¹ | 95% | 27% | No | 1-2 weeks |
| Guided | 95% | 55% | Yes | None |
| Therapeutics² | | | | |
| Pap + Guided | 95% | 65% | No | 1-2 weeks |
| Therapeutics¹ | | | | |
| 300 nm | 88-97% | 88-97% | Yes | None |
| Fluorescence | | | | |
| Excitation (In | | | | |
| vitro)⁴⁻⁵ | | | | |

¹Werner, c., W Griffith III, R Ashfaq, D Gossett, et. al., “Comparison of HPV Testing and Spectroscopy Combined with Cytology for the Detection of High-grade Cervical Neoplasia,” American Society for Colposcopy and Cervical Pathology Biennial Meeting, March, 2006 (http://www.guidedinc.com/Publications.htm), incorporated herein by reference.
²Chakhtoura, Nahida, et. al. “Quantitative Optical Spectroscopy of the Uterine Cervix: A cost effective way to detect and manage cervical disease,” INCOW, August 2007; see also http://www.guidedinc.com/Publications.htm, both incorporated herein by reference.
³Mitchell, Michele Follen, David Schottenfeld, Guillermo Tortolero-Luna, Scott B. Cantor, and Rebecca Richards-Kortum, “Colposcopy for the Diagnosis of Squamous Intraepithelial Lesions: A Meta-Analysis,” Obstet Gynecol 1998; 91(4)626-31, incorporated herein by reference.
⁴Glassman, W. Sha, C H Liu, G C Tang, S Lubics, and R R Alfano, “Ultraviolet Excited Fluorescence Spectra from Non-malignant and Malignant Tissues of the Gynecological Tract,” Lasers in the Life Sciences 1992; 5(1-2): 49-58.
⁵Huang, Z. Z., W. Sha. Glassman, G. C. Tang, S. Lubics, and R R Alfano, “Fluorescence Diagnosis of Gynecological Cancerous and Normal Tissues,” Proceedings of Advances in Laser and Light Spectroscopy to Diagnose Cancer and Other Diseases. SPIE 1994; 2135: 42-45.

Data collected by the experimental system require manipulation prior to evaluation. Data produced by the Hamamatsu spectrometer include detector noise. For low light level measurements, this detector noise forms a background that can swamp the signal. Prior to data collection, the Hamamatsu spectrometer is run in dark mode, measuring the detector electronic noise when the detector is shielded from light. This dark level is subtracted from the measured data prior to further data analysis.

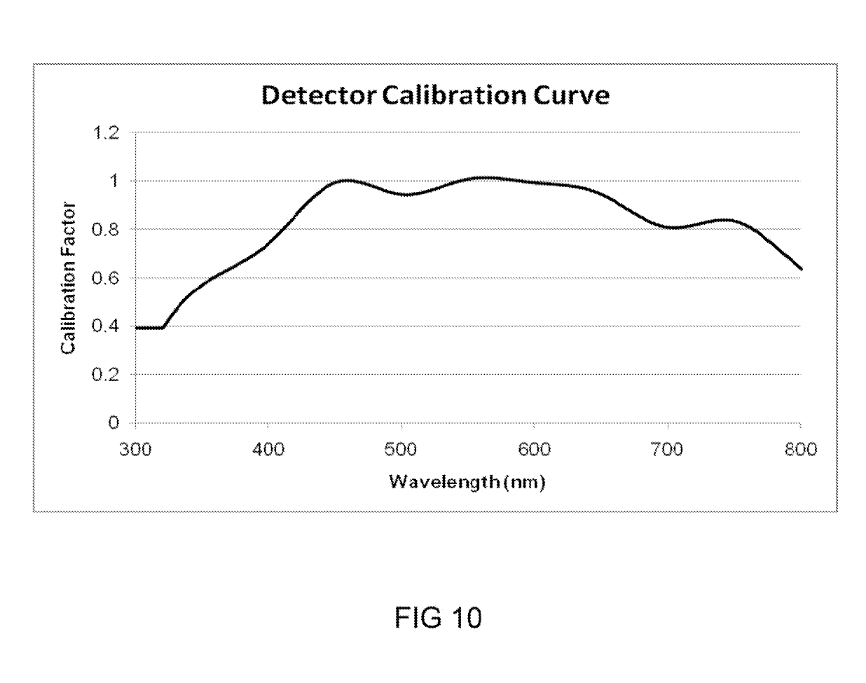

Following the dark subtraction, the data are divided by a spectral correction factor that accounts for the wavelength-dependent sensitivity of the CCD sensor. The spectral correction factor used in this study is shown graphically in FIG. 10. This calibration curve is specific to the silicon-based CCD sensor used in the experimental apparatus. Calibration curves of alternative shape and magnitude are possible, depending upon the specific detector or detectors used in the apparatus.

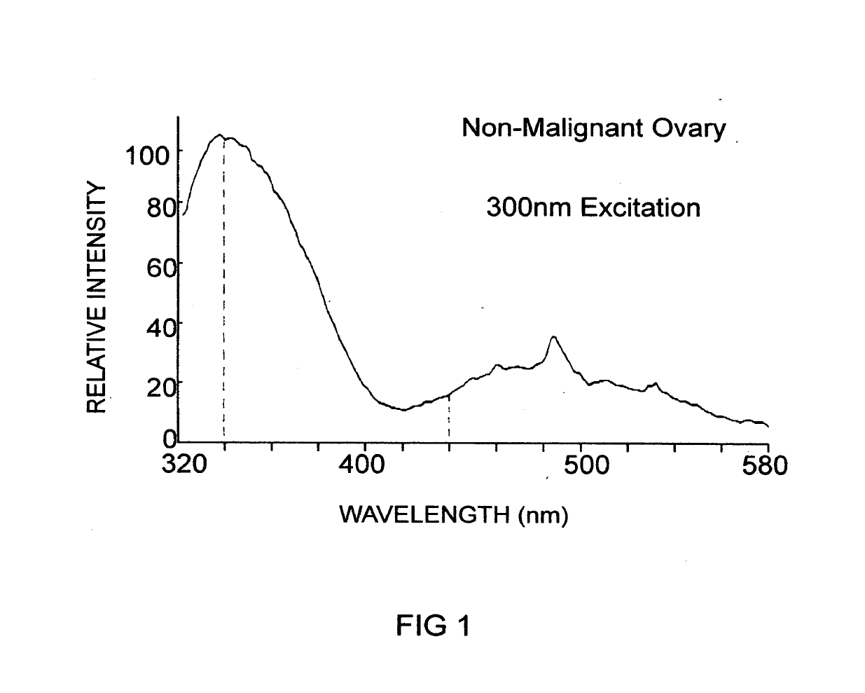

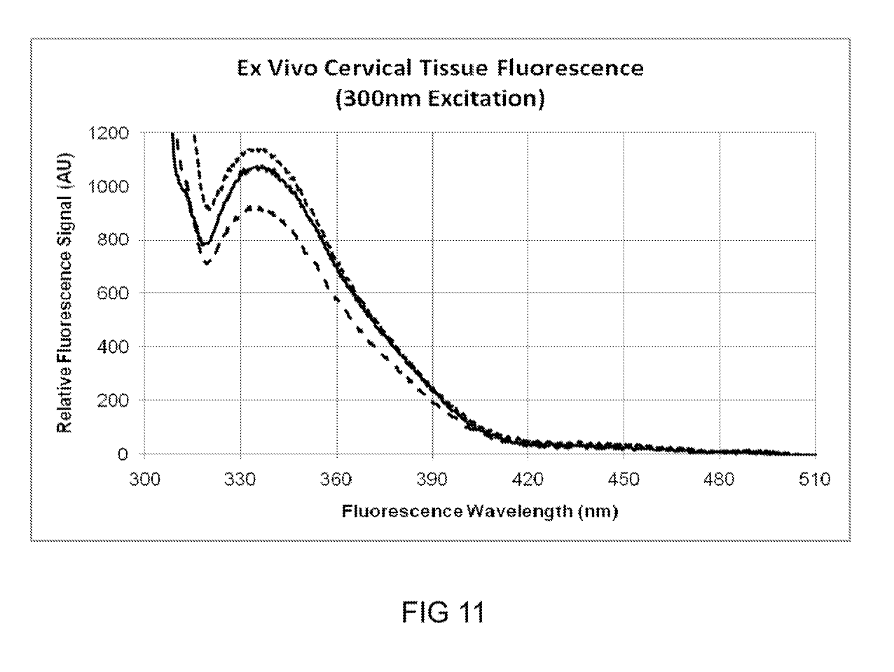

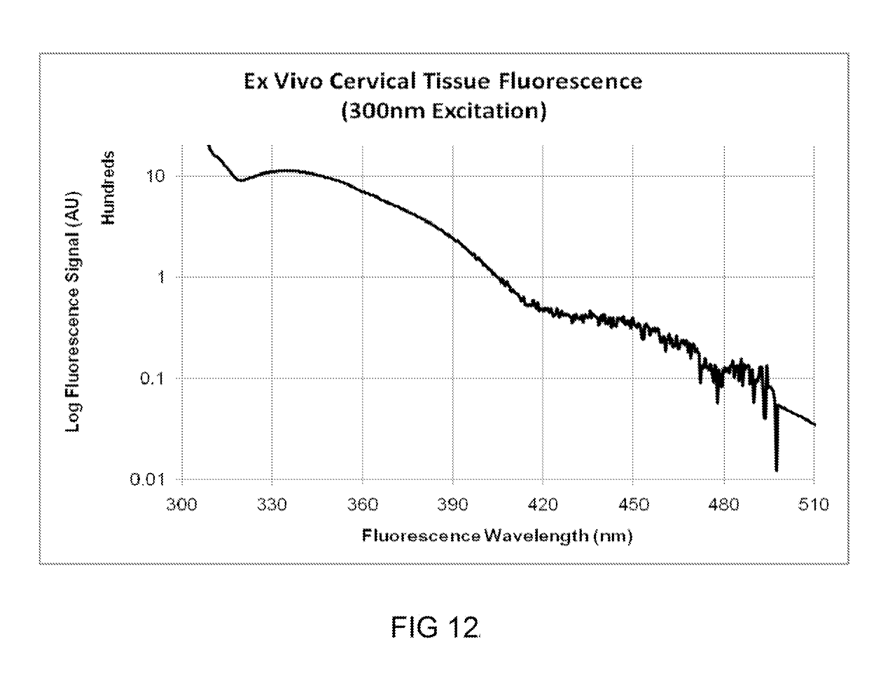

Data collected from human gynecological tissues using a point probe such as that depicted in FIG. 4 are shown in FIGS. 11 and 12. FIG. 11 illustrates the fluorescence collected from an ex vivo human cervical tumor, illuminated with light in the 290-310 nm band. The curves in FIG. 11 each have maxima (off scale) around 300 nm corresponding to the illumination signal that is reflected into the measurement fiber. A maximum is clearly evident in each of the plots around the tryptophan emission peak, with the basic shape of the fluorescence curve similar to that shown in FIG. 1, measured for ovarian tissue.

The dataset of FIG. 11 having the highest value around 340 nm is plotted again in FIG. 12, with the fluorescence amplitude shown using a log scale. The use of the log scale makes evident a series of subtle sub-peaks around 390 nm, 450 nm, and 490 nm, in addition to the primary peak around 340 nm. Comparison of the sub-peaks to the fluorophore emission spectra of FIG. 12 suggests that the feature at 390 nm is due to collagen/elastin, with that around 450 nm from NADH, and the 490 nm peak potentially due to flavins. The presence of these sub-peaks is consistent with published data of fluorescence from human tissues when illuminated with 300 nm light, and from the prior art data of Glassman and Huang, previously discussed.

The invention has been described in detail with particular reference to certain preferred embodiments thereof, but it will be understood that variations and modifications can be effected within the spirit and scope of the invention.

What is claimed is:

1. An apparatus for cervical cancer screening, comprising:
one or more light sources aligned with a beginning of a first optical test path and a beginning of a second optical test path;
one or more optical detectors aligned with an end of the first optical test path and an end of the second optical test path; and
a processor coupled to the one or more light sources and the one or more optical detectors, wherein
one of the first or second optical test paths comprises a moveable point probe positionable interior to the cervical canal that scans the endocervical canal by collecting fluorescence from successive patches of the endocervical canal.

2. The apparatus of claim 1, wherein the first optical test path is configured to interrogate an outer portion of a cervix, and the second optical test path is configured to interrogate an interior of a cervical canal.

3. The apparatus of claim 2, wherein the first optical test path comprises a window transmissive to light from the one or more light sources and transmissive to reflected light from the outer portion of the cervix which the first optical test path is configured to interrogate.

4. The apparatus of claim 2, wherein the first optical test path comprises a window transmissive to light from the one or more light sources and transmissive to fluorescence light from the outer portion of the cervix which the first optical test path is configured to interrogate.

5. The apparatus of claim 2, wherein the second optical test path comprises a window transmissive to light from the one or more light sources and transmissive to reflected light from the interior of the cervical canal which the second optical test path is configured to interrogate.

6. The apparatus of claim 2, wherein the second optical test path comprises a window transmissive to light from the one or more light sources and transmissive to fluorescence light from the interior of the cervical canal which the second optical test path is configured to interrogate.

7. (canceled)

8. The apparatus of claim 2, wherein the point probe comprises at least one side-directed fiber with the side-directed fiber cleaved at an angle around 40 degrees so that the illumination light is totally internally reflected at the fiber cleave.

9. The apparatus of claim 1, wherein the second optical path comprises at least a portion of the first optical path.

10. The apparatus of claim 9, further comprising a probe body and an attachment cap, wherein the probe body comprises the first optical path and wherein the attachment cap and the probe body comprise the second optical path.

11. (canceled)

12. A method for cervical cancer screening using two optical probes, comprising:
an imaging probe used to form an image primarily of the outer portion of the cervix;
a second probe comprising a moveable point probe positionable interior to the cervical canal that scans the endocervical canal by collecting fluorescence from successive patches of the endocervical canal.

13. The method of claim 12, wherein the point probe comprises at least one side-directed fiber with the side-directed fiber cleaved at an angle around 40 degrees so that the illumination light is totally internally reflected at the fiber cleave.

14. (canceled)

15. The method of claim 12, wherein the images of the outer portion of the cervix are formed from both reflected light and fluorescence emitted from the cervical tissues.

16. (canceled)

17. (canceled)

18. (canceled)

19. (canceled)

20. A method of screening the cervical canal for cervical cancer wherein one or more mechanisms are used to control the positioning of a moveable probe interior to the endocervical canal including
an interior screw element and an exterior screw body that mate.

21. The method of claim 20, wherein said one or more mechanisms are automated to control the exposure of the illumination light.

22. An apparatus to screen the cervical canal comprising a moveable optical fiber positionable interior to the cervical canal having a fiber rotation means and a longitudinal translation means including
an interior screw element and an exterior screw body that mate.

23. The apparatus of claim 22, wherein said apparatus to screen the cervical canal comprises a side-directed fiber cleaved at an angle around 40 degrees so that the illumination light is totally internally reflected at the fiber cleave.

24. The apparatus of claim 22 wherein control of the depth of insertion of the moveable optical fiber is done through the use of screw features or step features and translation means in mating tubes.

25. The apparatus of claim 22 further comprising an automated processor used to limit or to fix the exposure of the tissue under test to the illumination light.

26. The apparatus of claim 22 wherein the fiber rotation means and longitudinal translation means move the at least one optical fiber along the cervical canal through a predetermined pathway.

27. The apparatus of claim 22 wherein the fiber rotation means and longitudinal translation means move the at least one optical fiber along the cervical canal through a predetermined pathway in a motorized fashion.
